(19) United States
(12) Patent Application Publication
(10) Pub. No.: US 2013/0184789 A1
(43) Pub. Date: Jul. 18, 2013

(54) METHOD FOR NEUROMODULATION

(71) Applicant: ACANDIS GMBH & CO. KG, Pfinztal (DE)

(72) Inventors: Alfred Stett, Reutlingen (DE); A. Gharabaghi, Tuebingen (DE); Giorgio Cattaneo, Karlsruhe (DE)

(73) Assignee: ACANDIS GMBH & CO. KG, Pfinztal (DE)

(21) Appl. No.: 13/744,598

(22) Filed: Jan. 18, 2013

(30) Foreign Application Priority Data
Jan. 18, 2012 (DE) 10 2012 100 388.2

(51) Int. Cl. A61N 1/36

(52) U.S. Cl. CPC A61N 1/36
USPC 607/72

(57) ABSTRACT

The invention relates to a method for neuromodulation, in which an electrode comprising a compressible and expandable grid structure with cells formed from grid webs is arranged within a blood vessel, wherein the grid structure is expanded and, at least in expanded state, is exposed to electrical energy such that surrounding postganglionic parasympathetic nerve fibres of the sphenopalatine ganglion are electrically stimulated by the electrode.

### CROSS-REFERENCE TO RELATED APPLICATIONS

This application claims priority to German Patent Application No. 10 2012 100 388.2 filed Jan. 18, 2012 which is herein incorporated by reference.

### BACKGROUND OF THE INVENTION

1. Field of the Invention

The invention relates to a medical method for neuromodulation.

2. Background Art

With acute stroke (apoplexy), the blood supply to the brain cells is at least partially interrupted so that, in the affected areas of the brain, the cells are insufficiently supplied with oxygen and other nutrients. A thrombus that forms in larger blood vessels and is suddenly flushed into a smaller blood vessel in the brain often results in narrowing or occlusion of the smaller blood vessel.

However, within a specific time window, there is a possibility of preventing irreparable damage to the affected areas of the brain, i.e. a complete necrosis of brain cells. In practice, this usually entails the use of drug treatment, known as thrombolysis, during which, the patient is given a drug which dissolves the thrombus and hence restores the blood flow to the affected area of the brain.

Thrombolysis has several drawbacks. For example, it is known from the relevant specialist literature that the use of thrombolytic drugs is only effective in a time window of up to four and half hours after the event. However, it is also known that nerve cells are still able to regenerate for up to 48 hours after the event, i.e. that the consequences of stroke are reversible. In other words, thrombolytic drug treatment can only be performed efficiently in a fraction of the time actually available for the rescue of nerve cells. The by far greater portion of the available time window remains virtually unused in the case of drug treatment.

Since thrombolytic drugs influence blood clotting, there is also a risk of bleeding being induced in already damaged areas. In addition, the drug does not act locally on the affected areas of the brain, but is distributed throughout the whole body by the blood circulation, thus increasing the risk of side effects.

### SUMMARY OF THE INVENTION

An object of the invention is to provide a method for neuromodulation which is locally applicable and avoids the aforementioned drawbacks. According to the invention, these and other objects are achieved by a method for neuromodulation, in which an electrode comprising a compressible and expandable grid structure with cells formed from grid webs is arranged within a blood vessel, wherein the grid structure is expanded and, at least in an expanded state, is exposed to electric energy such that surrounding postganglionic parasympathetic nerve fibres of the sphenopalatine ganglion are electrically stimulated by the electrode.

### BRIEF DESCRIPTION OF THE DRAWINGS

FIG. 1a depicts a side view of an electrode for use in a preferred embodiment of the inventive method, wherein the electrode is arranged in a medially aligned segment of the internal carotid artery;

FIG. 1b depicts the electrode of FIG. 1a in a free arrangement;

FIG. 1c depicts a side view of an electrode for use according to a preferred embodiment of the inventive method in an implanted state depicting the electric field pattern;

FIG. 1d depicts a cross-sectional view through the electrode according to FIG. 1c; and

FIG. 2 depicts a side view of an electrode for use in a preferred embodiment, wherein the electrode is arranged in a cranially aligned segment of the internal carotid artery.

### DETAILED DESCRIPTION OF THE PREFERRED EMBODIMENT(S)

The invention is based on a method for neuromodulation, in which an electrode comprising a compressible and expandable grid structure with cells formed from grid webs is arranged within a blood vessel, wherein the grid structure is expanded and, at least in expanded state, is exposed to electrical energy such that surrounding postganglionic parasympathetic nerve fibres of the sphenopalatine ganglion are electrically stimulated by the electrode.

Unlike known drug treatment aimed at the dissolution of a thrombus, the electrical stimulation of nerve fibres in the method according to the invention causes blood vessels to expand. In other words, electrical stimulation of the nerve fibres causes vasodilatation, in particular of cerebral arteries and/or arterioles. At least temporarily, an enlarged flow cross section is provided so that the affected areas of the brain are supplied with oxygen and nutrients once again.

Generally, the grid structure of the electrode can be embodied as a hollow cylinder, in particular as a stent. The grid structure is preferably self-expanding.

The grid structure can be made of one single material, which is electrically conductive. It can also be provided that the grid structure is coupled to a power supply by an electric line, in particular at least one litz wire.

The grid structure of the electrode can also comprise at least one electrically conductive layer and at least one mechanically supporting layer. The electrically conductive layer and the mechanically supporting layer can be made of different materials.

It can also be provided that the electrically conductive layer is arranged externally for contact with the blood vessel and the mechanically supporting layer is arranged radially further inwardly relative to the electrically conductive layer.

In a preferred embodiment of the method, the electrode is introduced into the blood vessel via a microcatheter. The microcatheter can be guided to the blood vessel via a peripheral blood vessel, in particular the femoral artery.

It is particularly preferred if the electrode is expanded in the internal carotid artery and exposed to the electrical energy. The electrical energy can be pulsed.

The above-described method is particularly suitable for stroke treatment, in particular by electrical stimulation of postganglionic parasympathetic nerve fibres of the sphenopalatine ganglion. The invention therefore also relates to the use of the above-described method for stroke treatment.

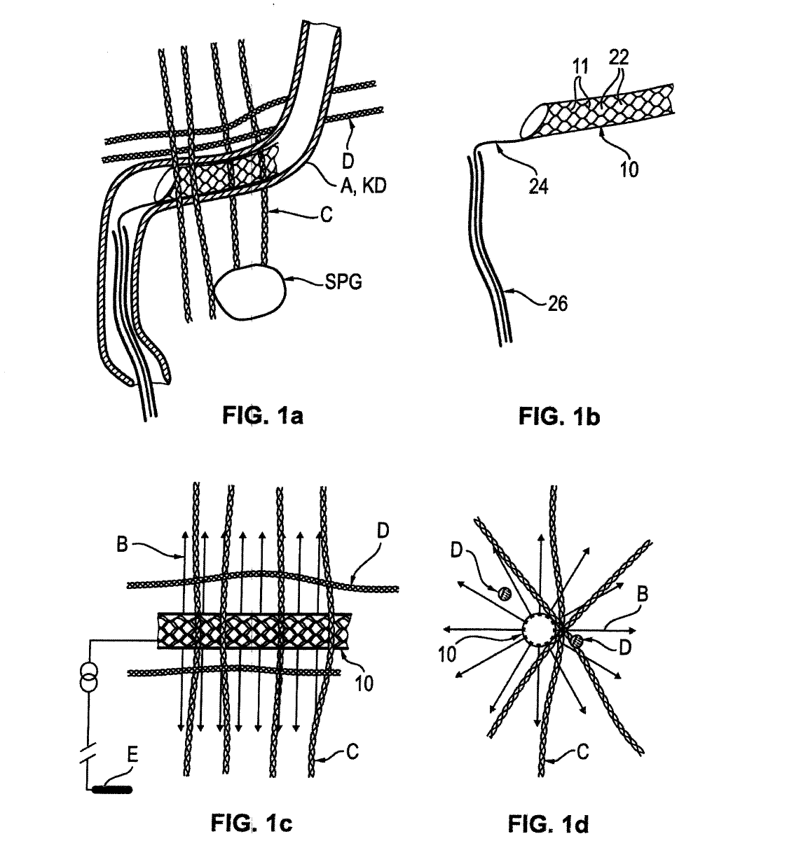

The electrode for endovascular medical applications shown in FIG. 1a is in particular suitable for the stimulation of nerves located in the immediate vicinity of cavities or hollow organs or a blood vessel A. Particularly advantageously, the electrode is used for the treatment of acute ischaemic stroke (apoplexy). Here, the objective is to activate the postganglionic parasympathetic nerve fibres of the sphenopalatine ganglion in a minimally invasive and selective way by means of endovascular, deep brain stimulation. A microcatheter carrying the electrode is introduced via a peripheral blood vessel in the groin, in particular the femoral artery. The microcatheter and the electrode are guided into the carotid artery, in particular the internal carotid artery, where the electrode is released.

The electrode comprises a grid structure 10, preferably embodied as an open stent structure. Therefore, the grid structure 10 comprises a substantially hollow-cylindrical body with open longitudinal ends. The wall of the hollow-cylindrical body is formed by a grid or network comprising a plurality of grid webs 11 connected at points of intersection. The webs border cells 22 of the grid structure 10. During the electrical stimulation, which can be performed for a plurality of minutes, in particular a plurality of hours, the flow can pass through the open stent structure. Hence, the formation of a new thrombus can be advantageously avoided.

The stent structure, or generally the grid structure 10, is coupled to an electric line connecting the grid structure 10 or the electrode to a power supply. The grid structure 10 preferably has a fine-meshed structure to generate homogeneous electrical fields.

Due to the anatomical proximity of the internal carotid artery, in which the endovascular stimulation electrode, in particular the grid structure 10, is arranged, to the postganglionic parasympathetic nerve fibres of the sphenopalatine ganglion, selective activation is possible, which, in addition to the interventional access via the groin, also enables minimally invasive treatment. Many stroke patients commonly have catheter access in the groin in for the administration of drugs, for example thrombolytic agents, so that further invasive interventions for the treatment with the electrode are avoided.

The stimulation of the nerve fibres of the SPG achieves inter alia vasodilatation of the arteries or arterioles in the intracerebral region. This effect can in particular be used in the treatment of stroke, wherein the rescue of damaged brain areas can take place by means of the provision of increased perfusion, i.e. blow flow or blood supply. Stimulation can also increase the permeability of the vessel wall and hence enable the administration of specific drugs.

The claimed method has two main advantages:
the electrode is administered in an interventional way via vascular access and therefore in a minimally invasive way—this reduces the risk of infection and injury, increases the speed of the treatment and improves acceptance on the part of the patient. A suitable catheter access in the groin is already present in many stroke patients for the administration of drugs, in particular thrombolytic agents (e.g. fibrinolytic agents, recombinant tissue plasminogen activators (rt-PA)), and so, in these cases, the treatment can be performed without any further interventions.
stimulation of the neuronal efferences of the SPG (instead of the ganglion itself) greatly increases the selectivity of the treatment. It is known that nerves extend from the ganglion in different directions and influence different physiological functions. These nerves stimulate, inter alia, the lacrimal and nasal glands. Stimulation of solely the nerves influencing cranial blood vessels results in selective vasodilation without any other physiological functions being disrupted.

For the selective stimulation of the postganglionic parasympathetic nerve fibres of the sphenopalatine ganglion (SPG), it is expedient for the electrode or stent electrode to be arranged in a precisely defined region of the circulation.

The stent electrode is preferably positioned in the internal carotid artery (ICA). The ICA has a so-called petrous segment along which the parasympathetic greater petrosal nerve extends. The petrous segment extends in the medial direction and therefore relatively perpendicular to the neuronal efferences of the SPG extending in the cranial direction. Particularly suitable for use in the petrous segment is an electrode with a grid structure 10, which generates a radially outwardly directed field. This means that nerves extending parallel to the vessel, for example the optical nerve, are not stimulated. This selective nerve stimulation can be clearly seen in FIGS. 1a to 1d.

FIG. 1a shows the arrangement of the electrode or the grid structure 10 in the internal carotid artery for the stimulation of the corresponding nerve fibres. The grid structure 10 is guided by the central supply line 24 embodied as a guide wire through the microcatheter 26 into the internal carotid artery where it is released from the microcatheter 26. This causes the grid structure 10 to expand automatically, i.e. the grid structure is self-expanding. In addition, a pulsed current is supplied via the central supply line 24 into the grid structure 10 thus effecting electrical stimulation of the nerve fibres. The electrical stimulation of the nerve fibres causes the blood vessels, in particular the intracerebral vessels, to expand so that cerebral circulation is increased.

The electrode or grid structure 10 is preferably embodied as retractable. To this end, as shown in FIG. 1b, an axial end of the grid structure 10 can comprise an oblique smooth edge so that, on the advancement of the microcatheter 26 through the central supply line 24, the grid structure 10 is compressed as soon as the tip of the microcatheter 26 slides along the oblique end edge of the grid structure 10.

FIGS. 1c and 1d show the gradient of the field lines of the electrical field emanating from the grid structure 10 of the electrode which interacts with an extracorporally arranged second electrode (not shown). In the environment of the blood vessel A, nerve fibres extend parallel to the blood vessel A on the one hand and orthogonal thereto on the other. The electrode, in particular a stent electrode, substantially represents a cylindrical electrode, wherein the field lines B of the electrical field extend substantially perpendicularly to the surface or outer circumference of the grid structure 10. This causes the nerve fibres C extending orthogonally to the axis of the grid structure 10, i.e. orthogonally to the blood vessel A, to be stimulated. The nerve fibres D extending in parallel to the blood vessel A experience substantially no stimulation from the electrical field of the electrode.

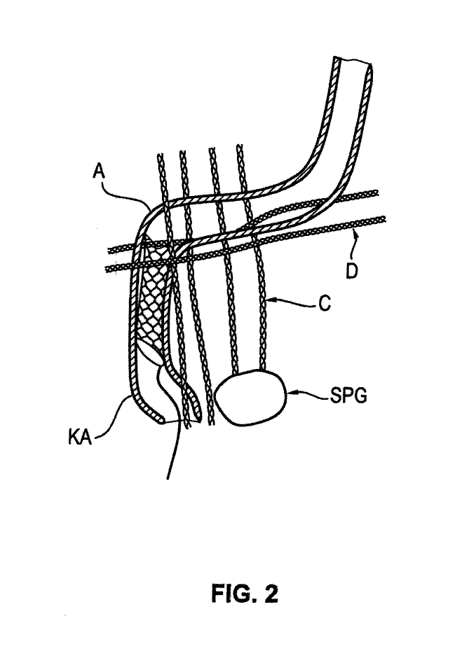

However, it is also possible to position the electrode, in particular the grid structure 10, in the proximal segment of the ICA, extending in the cranial direction, as shown in FIG. 2. In this case, the electrode is preferably exposed to electric voltage such that a field extending parallel to the axis of the electrode is formed. Advantageously, the electrode is bipolar, i.e. the grid structure has two electric poles. Preferably, the poles are embodied on the longitudinal ends of the grid structure. Alternatively, two electrodes insulated from each other can be positioned in sequence, each forming one of the two poles in order to achieve the same effect.

Preferably, the electrode is a part of a system, which, in addition to a power supply, comprises a pulse generator, control electronics and, optionally, a counter-electrode E. The counter-electrode E can be embodied similarly to the electrode. Alternatively, the counter-electrode E can be an extracorporal electrode arranged outside of the body on the patient's skin. The other electrode or counter-electrode can also be formed from the tip of the microcatheter 26 or a guide wire. The electric contact between the counter-electrode E and a power supply is provided by a line arranged in the walls of the catheter between two plastic layers. The line can be a wire extending in the axial direction along the microcatheter. Alternatively, an existing coil or a braid supporting the microcatheter can be used as the line. In conjunction with a counter-electrode, the system is particularly suitable for signal acquisition of electric cell activities. A comparative measurement between the electrode and the counter-electrode E enables electric potentials within the body to be measured and used for further therapeutic measures. Insofar, the system or the electrode can be used as a sensor. Preferably, the system or the electrode can be used for the acquisition of nerve signals. It is possible for the system to be used on the one hand for stimulation, that is to output electric signals into body tissue, and on the other for signal acquisition, i.e. for the determination of electric signals from the body tissue. It is possible to switch between the two functions (stimulation and signal acquisition) manually or automatically.

Further fields of application of the electrode for endovascular medical applications relate to the treatment of migraine, Parkinson's disease, epilepsy, depression, compulsive behaviour or general therapeutic methods relating to deep brain stimulation. It is also conceivable to use the electrode in connection with high blood pressure (hypertension) for example in renal vessels or carotid arteries.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for neuromodulation, comprising arranging an electrode comprising a compressible and expandable grid structure with cells formed from grid webs within a blood vessel, expanding the grid structure and, at least in an expanded state, exposing the grid structure to electric energy such that surrounding postganglionic parasympathetic nerve fibres of the sphenopalatine ganglion are electrically stimulated by the electrode.

2. The method of claim 1, wherein the grid structure comprises at least one electrically conductive layer and at least one mechanically supporting layer, the electrically conductive layer and mechanical supporting layer optionally made of different materials.

3. The method of claim 2, wherein the electrically conductive layer is arranged externally for contact with the blood vessel and the mechanically supporting layer is arranged radially further inwardly relative to the electrically conductive layer.

4. The method of claim 1, wherein the electrode is introduced into the blood vessel via a microcatheter, wherein the microcatheter is guided to the blood vessel via a peripheral blood vessel.

5. The method of claim 2, wherein the electrode is introduced into the blood vessel via a microcatheter, wherein the microcatheter is guided to the blood vessel via a peripheral blood vessel.

6. The method of claim 3, wherein the electrode is introduced into the blood vessel via a microcatheter, wherein the microcatheter is guided to the blood vessel via a peripheral blood vessel.

7. The method of claim 1, wherein the electrode is introduced into the blood vessel via a microcatheter, wherein the microcatheter is guided to the blood vessel via the femoral artery.

8. The method of claim 1, wherein the grid structure of the electrode is expanded in the internal carotid artery and therein exposed to the electric energy.

9. The method of claim 2, wherein the grid structure of the electrode is expanded in the internal carotid artery and therein exposed to the electric energy.

10. The method of claim 3, wherein the grid structure of the electrode is expanded in the internal carotid artery and therein exposed to the electric energy.

11. The method of claim 4, wherein the grid structure of the electrode is expanded in the internal carotid artery and therein exposed to the electric energy.

12. The method of claim 7, wherein the grid structure of the electrode is expanded in the internal carotid artery and therein exposed to the electric energy.

13. The method of claim 1, wherein the electric energy is pulsed.

14. The method of claim 2, wherein the electric energy is pulsed.

15. The method of claim 3, wherein the electric energy is pulsed.

16. The method of claim 4, wherein the electric energy is pulsed.

17. The method of claim 7, wherein the electric energy is pulsed.

18. A method for the treatment of stroke, comprising employing the method of claim 1.
